(19) United States
(12) Patent Application Publication
(10) Pub. No.: US 2023/0191098 A1
(43) Pub. Date: Jun. 22, 2023

(54) STRAP FOR MICRONEEDLE APPLICATOR

(71) Applicant: JUBILEE BIOTECH CO., LTD., Wonju-si (KR)

(72) Inventors: Sung Kyoung LEE, Seoul (KR); Goonghyun HAN, Siheung-si (KR)

(73) Assignee: JUBILEE BIOTECH CO., LTD., Wonju-si (KR)

(21) Appl. No.: 17/559,973

(22) Filed: Dec. 22, 2021

(51) Int. Cl. A61M 37/00

(52) U.S. Cl. CPC A61M 37/0015; A61M 2037/0023; A61M 2209/088

(57) ABSTRACT

A strap for a microneedle applicator according to the present disclosure, which is coupled to an applicator main body part of the microneedle applicator in the form of a wearable device to allow the microneedle applicator to be worn on the body, includes a strap part having a structure that is inflatable when air is injected thereinto, a main body connecting part installed at both ends of the strap part, a housing installed at one side of an outer surface of the strap part, a pump installed inside the housing and configured to supply air to be injected into the strap part, a valve installed inside the housing, connected to a communication path along which air is injected into the strap part, and configured to operate to inject air into the strap part or discharge air from inside the strap part to the outside, and a controller configured to control the pump and the valve.

### BACKGROUND

### 1. Field of the Invention

The present disclosure relates to a strap for a microneedle applicator, and more particularly, to a strap for a microneedle applicator that has a function of bringing an applicator main body into close contact with the skin so that, when the skin is punctured with a microneedle through the microneedle applicator, puncturing with the microneedle is more efficiently performed.

### 2. Discussion of Related Art

Generally, infusing a drug into the skin is referred to as a transdermal drug delivery system. Since the transdermal drug delivery system does not involve the gastrointestinal tract, a drug can be administered regardless of the acidity of the gastrointestinal tract, enzymes in the gastrointestinal tract, food in the gastrointestinal tract, and movement of the gastrointestinal tract.

For the transdermal drug delivery system, ultrasonic waves, jet injection, electroporation, iontophoresis, hypodermic needles, chemical penetration enhancers, microneedles, and the like are used.

Microneedles having a length of hundreds of micrometers deliver a drug component into the skin through the stratum corneum of the skin. A microneedle was developed by Mark Prausnitz in 1998 as a next-generation drug delivery system that combines a conventional syringe with the convenience of a patch to eliminate the fear of needles.

In addition to being able to deliver macromolecular substances such as proteins and peptides, microneedles have advantages such as enabling painless drug delivery, allowing faster recovery of the administration site as compared to general infusions, having a low risk of contamination and infection, and, due to their high effectiveness, allowing the amount of administered drug to be reduced. Accordingly, much research and development for the application of microneedles has taken place.

At an early stage, solid-type microneedles that form fine holes in the skin and allow a drug to penetrate into the skin through the formed holes were widely used. Such solid-type microneedles are still widely used by being processed into the form of a roller in the cosmetic industry.

Microneedles for subcutaneous infusion of drugs are classified into a coated type in which a surface of a needle is coated with a drug, a dissolving type in which a needle itself is formed with a material containing a drug component and the needle is dissolved in the skin, and a hollow type in which a drug is infused through a hollow inside a needle.

U.S. Pat. No. 8,668,675 discloses an applicator that punctures the skin with the hollow-type microneedle to infuse a drug.

The coated-type or dissolving-type microneedle is often manufactured as a patch type, and such a microneedle patch is attached to the skin by hand or by an applicator disclosed in U.S. Pat. No. 10,035,008.

The patch-type and hollow-type microneedles are both for one-time use only and thus have a problem in that periodical or repeated infusion is not possible.

While one infusion is sufficient for medicines such as vaccines, most drugs require repeated administration at predetermined time intervals. An applicator for the hollow-type microneedle lacks such a function, and an applicator for the patch-type microneedles has an inconvenience of requiring an attached patch to be replaced at predetermined time intervals.

Through International Unexamined Patent Application Publication No. WO 2021/167410, the present applicant has disclosed a microneedle applicator and a cartridge that allow a drug to be periodically infused using a microneedle as illustrated in FIGS. 1 to 5.

A microneedle applicator 1 previously filed by the present applicant is a wearable device in the form of a watch. The previously-filed microneedle applicator 1 has an applicator main body 200 having a cartridge 100, which has a plurality of microneedle bases 151 installed therein, mounted thereon and presses one of the microneedle bases 151 toward the skin using a pressing end 404 to puncture the skin with a microneedle 153 disposed at a lower surface of the microneedle base 151 and infuse a drug into the skin.

The cartridge 100 has a plurality of through-holes 150 formed therein, and an inner wall of the through-hole 150 and the microneedle base 151 are connected by a connecting member 155.

The pressing end 404 of the previously-filed microneedle applicator operates by an interaction between a driving source 402 formed of a motor and a power transmitter 403 formed of a gear. Also, the pressing end 404, the driving source 402, and the power transmitter 403 integrally rotate about an operation shaft 450. Due to the rotation, the pressing end 404 moves to above one of the microneedle bases 151, which will infuse a drug, and then moves downward to press the microneedle base 151 toward the skin. Accordingly, the skin is punctured with the microneedle 153 disposed at the lower surface of the microneedle base 151, and the drug is infused into the skin. When drug infusion is completed, the pressing end 404 moves upward, and the microneedle base 151 returns to its original position due to elasticity of the connecting member 155.

In addition to the microneedle applicator 1 previously filed by the present applicant, the applicator according to U.S. Pat. No. 10,035,008 presses a microneedle toward the skin using a pressing member inside the applicator in a state in which the applicator is brought into close contact with the skin.

For the skin to be easily punctured with the microneedle, the applicator should come in contact with the skin firmly, at a predetermined level or more.

Also, the related arts relating to pressing a main body through a strap in a wearable device can be found in wrist-worn sphygmomanometers.

U.S. Pat. No. 4,307,727 discloses adjusting tension of a strap through a cam, U.S. Pat. No. 4,896,676 discloses causing a strap to inflate due to air injected through a manual pump, and U.S. Patent Publication No. 2020/0085319 discloses injecting air into a bladder behind a strap using a micro pump.

Blood pressure devices that inflate a strap supply air using a manual or automatic pump in a main body connected to the strap. Thus, there is a problem in that, when various different parts are required to be embedded in the main body, the size of the main body increases.

### SUMMARY OF THE INVENTION

The present disclosure is directed to providing a strap capable of pressing an applicator main body toward the skin to facilitate puncturing the skin with a microneedle.

The present disclosure is also directed to providing a strap having a structure capable of securing a necessary space inside an applicator main body by causing a structure configured to inflate the strap to be disposed outside the applicator main body.

The present disclosure is also directed to providing a strap capable of adjusting a pressed portion of an applicator main body and a pressing force.

A strap for a microneedle applicator according to the present disclosure, which is coupled to an applicator main body part of the microneedle applicator in the form of a wearable device to allow the microneedle applicator to be worn on the body, includes a strap part having a structure that is inflatable when air is injected thereinto, a main body connecting part installed at both ends of the strap part, a housing installed at one side of an outer surface of the strap part, a pump installed inside the housing and configured to supply air to be injected into the strap part, a valve installed inside the housing, connected to a communication path along which air is injected into the strap part, and configured to operate to inject air into the strap part or discharge air from inside the strap part to the outside, and a controller configured to control the pump and the valve.

The controller may be disposed inside the applicator main body part or the housing.

Power or a signal necessary for the pump and the valve to operate may be transmitted through the main body connecting part.

In the strap part, a portion of a side that comes in contact with the body may be a thin film portion formed with a small thickness.

The thin film portion may be provided as a plurality of thin film portions.

Thicknesses or sizes of the plurality of thin film portions may be different from each other.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating an exterior of a microneedle applicator according to the related art;

FIG. 2 is a view illustrating the microneedle applicator and a cartridge according to the related art;

FIG. 3 is a view for describing an upper configuration of the cartridge according to the related art;

FIG. 4 is a view for describing a lower configuration of the cartridge according to the related art;

FIG. 5 is a view for describing the operational relationship of the microneedle applicator according to the related art;

FIG. 6 is a perspective view illustrating the form of a microneedle applicator having a strap mounted thereon according to an embodiment of the present disclosure;

FIG. 7 is a perspective view illustrating the strap according to an embodiment of the present disclosure;

FIG. 8 is a cross-sectional view of the strap according to an embodiment of the present disclosure;

FIG. 9 is a cross-sectional view when air is injected into the strap of FIG. 8; and

FIG. 10A and FIG. 10B are cross-sectional views illustrating configurations of a pump and a valve according to the present disclosure.

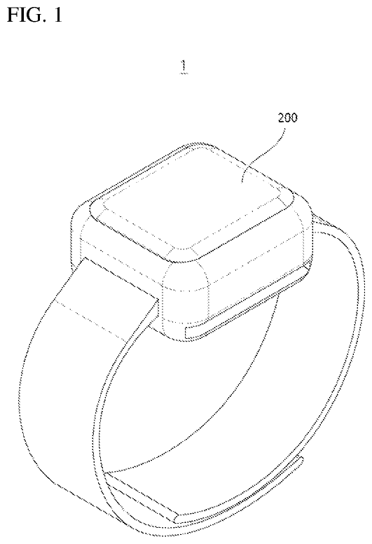

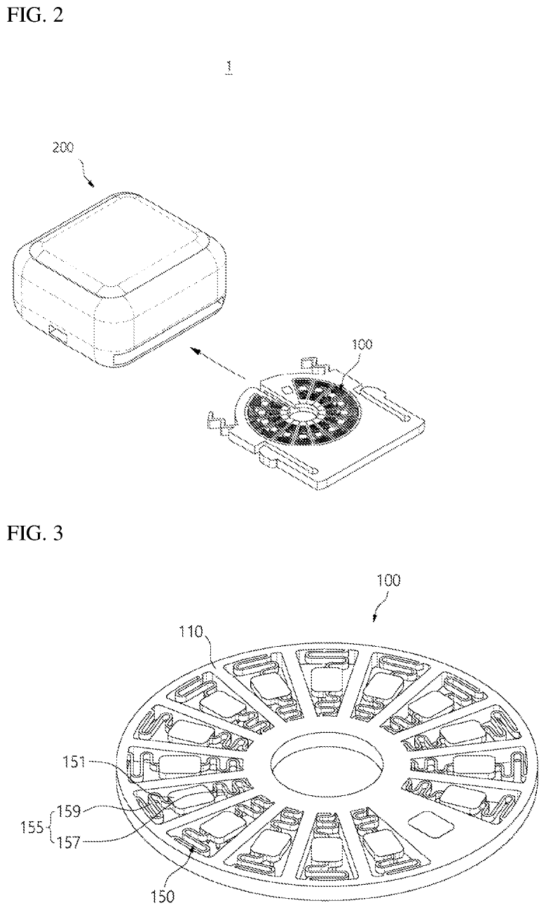

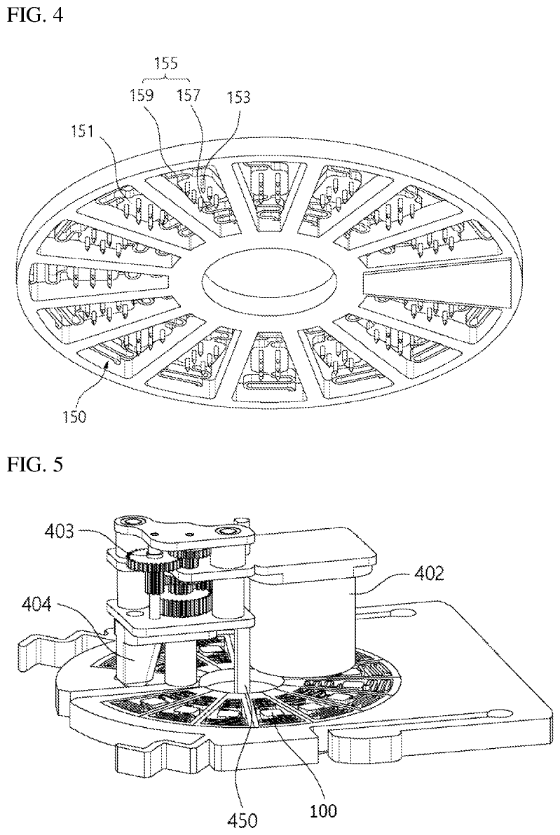

### DETAILED DESCRIPTION OF EXEMPLARY EMBODIMENTS

Hereinafter, embodiments of the present disclosure will be described in detail with reference to the accompanying drawings.

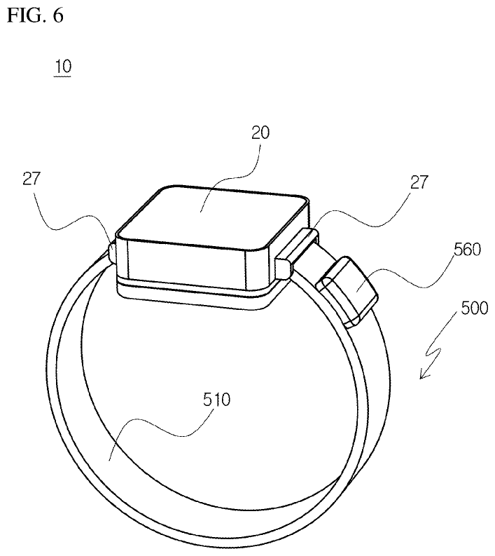

FIG. 6 illustrates a microneedle applicator having a strap mounted thereon according to an embodiment of the present disclosure.

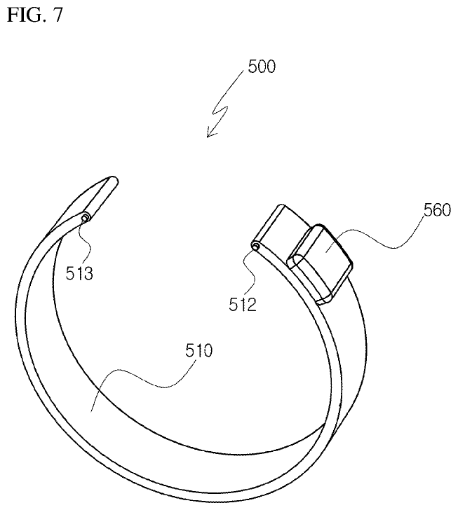

As illustrated in FIG. 7, a strap 500 is connected to a strap mounting part 27 of an applicator main body part 20 through main body connecting parts 512 and 513 disposed at both ends of the strap 500.

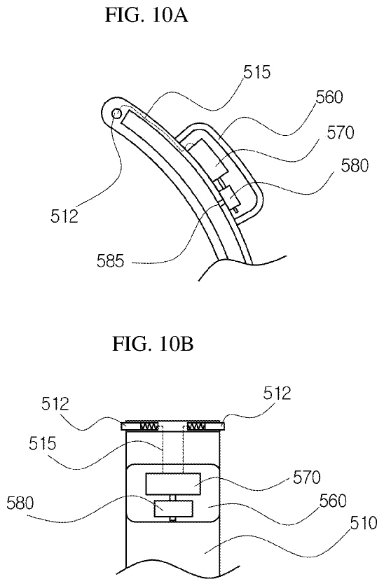

Each of the main body connecting parts 512 and 513 may be a pin installed in a form compressible in a longitudinal direction as in a strap of a general wristwatch or may include two separate pins (see FIG. 10B) that are separated in the longitudinal direction and are compressible.

A strap part 510 has a structure that is inflatable when air is injected thereinto. The strap part 510 may be made of one or more elastic materials such as silicone, synthetic resin, and rubber.

A housing 560 is disposed at one side of an outer surface of the strap part 510, and a pump 570 and a valve 580 are installed inside the housing 560. The housing 560 may be made of a different material from the strap part 510 or made of the same material as the strap part 510. Also, by performing insert-molding of the pump 570 and the valve 580 during injection molding of the strap part 510, the strap part 510 and the housing 560 may be integrally formed. In the present disclosure, the housing 560 refers to a space in which the pump 570 and the valve 580 are disposed.

The pump 570 pumps air to be injected into the strap 500 and delivers the pumped air to the valve 580, and the valve 580 is provided in the form of a three-way valve that is able to inject the air supplied from the pump 570 into the strap part 510 through a communication path 585 or is able to be opened to discharge air from inside the strap part 510.

The pump 570 and the valve 580 may receive power from the applicator main body part 20 through the main body connecting part 512. The strap mounting part 27 and the main body connecting par 512 of the microneedle applicator may constitute a connector structure capable of carrying current and may receive power and a signal necessary for operation through a signal line 515 that passes through the inside of the strap part 510.

Of course, a separate battery for operating the pump 570 and the valve 580 may be mounted on an outer portion of the strap.

A controller configured to control the operation of the pump 570 and the valve 580 may be disposed in the applicator main body part or mounted inside the housing 560.

When the controller is disposed inside the applicator main body part, a signal from the controller or power necessary for operation may be transmitted to the pump 570 and the valve 580 through the main body connecting part 512 or a separate wire.

When air is injected into the strap part 510 by the pump 580, the strap part 510 inflates, and the applicator main body part comes into contact more firmly with a site of the body where the microneedle applicator is worn, such as a wrist or a forearm.

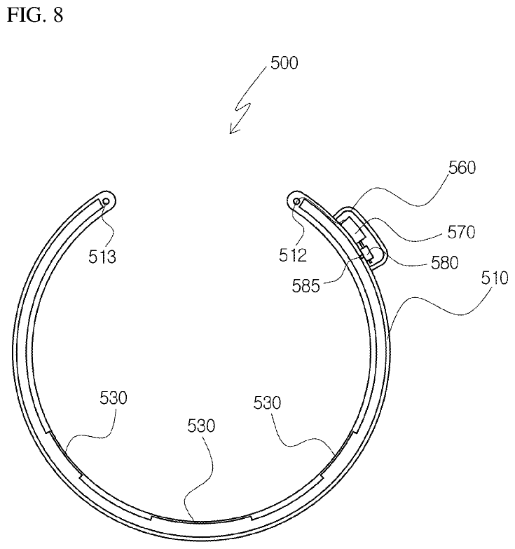

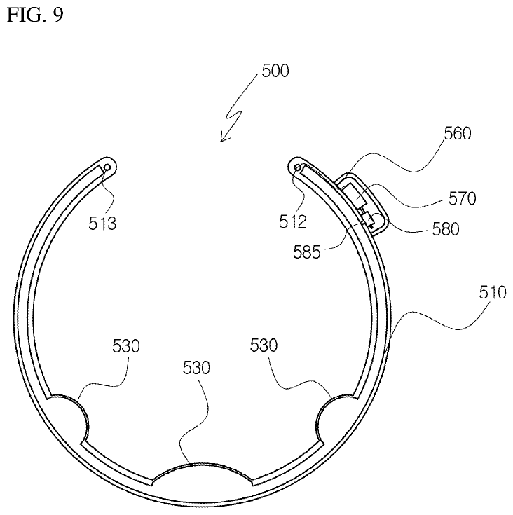

As illustrated in FIGS. 8 and 9, in the strap part 510, a portion of a side that comes in contact with the skin may be a thin film portion 530 formed to have a smaller thickness than surrounding portions.

The thin film portion 530 may be provided as a plurality of thin film portions 530, and thicknesses of the thin film portions 530 may be different from each other. In this case, the extent to which the thin film portion 530 inflates becomes different for each thin film portion 530, and according to such differences in the extent of inflation, a partial region of a lower surface of the microneedle applicator may be adjusted to come in contact more firmly with a wrist or a forearm.

The strap 500 according to the present embodiment is formed of a single strap part 510, but the strap part 510 may also be divided into two separate strap parts. When two strap parts are present, air pressure due to the pump and the valve may be supplied only to one strap part.

A strap for a microneedle applicator according to the present disclosure facilitates puncturing the skin with a microneedle, allows a free space to be secured inside an applicator main body, and allows a pressed portion of the applicator main body and a pressing force to be adjusted.

The above description is only for describing the technical spirit of the present disclosure by way of example, and various modifications and changes may be made by those of ordinary skill in the art to which the present disclosure pertains within the scope not departing from essential characteristics of the present disclosure.

Therefore, the embodiments disclosed herein are for describing, instead of limiting, the technical spirit of the present disclosure, and the scope of the technical spirit of the present disclosure is not limited by the embodiments. The scope of the present disclosure should be interpreted by the claims below, and all technical spirits within the scope equivalent thereto should be interpreted as falling within the scope of the present disclosure.

What is claimed is:

1. A strap for a microneedle applicator that is coupled to an applicator main body part of the microneedle applicator in the form of a wearable device to allow the microneedle applicator to be worn on the body, the strap comprising:
a strap part having a structure that is inflatable when air is injected thereinto;
a main body connecting part installed at both ends of the strap part;
a housing installed at one side of an outer surface of the strap part;
a pump installed inside the housing and configured to supply air to be injected into the strap part;
a valve installed inside the housing, connected to a communication path along which air is injected into the strap part, and configured to operate to inject air into the strap part or discharge air from inside the strap part to the outside; and
a controller configured to control the pump and the valve.

2. The strap of claim 1, wherein the controller is disposed inside the applicator main body part or the housing.

3. The strap of claim 1, wherein power or a signal necessary for the pump and the valve to operate is transmitted through the main body connecting part.

4. The strap of claim 1, wherein, in the strap part, a portion of a side that comes in contact with the body is a thin film portion formed with a small thickness.

5. The strap of claim 4, wherein the thin film portion is provided as a plurality of thin film portions.

6. The strap of claim 5, wherein thicknesses or sizes of the plurality of thin film portions may be different from each other.
